(19) United States
(12) Patent Application Publication
(10) Pub. No.: US 2010/0183526 A1
(43) Pub. Date: Jul. 22, 2010

(54) TIGER NUT MILK BASE CONCENTRATE AND METHOD FOR OBTAINING A COSMETIC PRODUCT FROM SAID BASE CONCENTRATE

(75) Inventors: Daniel Tortajada Forner, Alboraya (Valencia) (ES); Jose Antonio Novejarque Conde, Alboraya (Valencia) (ES)

Correspondence Address: WENDEROTH, LIND & PONACK, L.L.P., 1030 15th Street, N.W.,, Suite 400 East, Washington, DC, 20005-1503, US

(73) Assignee: Horchateria Daniel S.L.

(21) Appl. No.: 12/452,804

(22) PCT Filed: Jul. 24, 2008
(86) PCT No.: PCT/ES2008/000515

§ 371 Date: Mar. 17, 2010

(30) Foreign Application Priority Data
Jul. 24, 2007 (ES) P200702058

(51) Int. Cl. A61K 8/97; A61Q 17/04; A61K 8/99

(52) U.S. Cl. 424/59; 424/776; 424/195.15; 424/748; 424/750; 424/765; 424/735; 424/729

(57) ABSTRACT

The present invention relates to a method for obtaining a cosmetic product from a tiger nut milk concentrate, characterized in that it comprises:
a) mixing in a tank sugar-free tiger nut milk with cosmetic ingredients and thermal deionized distilled water;
b) mixing in a second tank surfactants with molasses alcohol;
c) mixing the products resulting from the previous steps in order to produce a base concentrate;
d) applying ultrasounds to said base concentrate; and
f) adding pharmaceutically acceptable excipients,
   as well as to the base concentrate, and to the cosmetic product resulting from said method.

### TECHNICAL FIELD OF THE INVENTION

The present invention is included in the field of the preparation of cosmetic products, more specifically, cosmetic products based on tiger nut milk concentrate.

### STATE OF THE ART PRIOR TO THE INVENTION

Tiger nut is planted on the months of April and May. The harvest is carried out between the months of November and January.

Once the harvesting process is finished, a wash thereof for cleaning and impurities removal is carried out. Next, a deep cleaning process for undesirable elements (straw and stones from the country) is applied. Subsequently the down is removed and a fine water shower for finishing the cleaning is provided.

Once the tiger nut is clean, the moisture level is then reduced by drying it. In this process is necessary to achieve a moisture reduction from 50% to 15%. Said drying time lasts about three months and is carried out in drying chambers in a slow and careful manner.

Once it is dried, its cleaning and sorting is then carried out in order to separate the impurities, the faulty or those of small size.

Once this cleaning and sorting operation has been carried out, they are packaged in sacs for its use.

From the tiger nuts thus obtained, the natural tiger nut milk is produced, this being a method common with that employed for subsequently preparing the liquid food typical from the Valencia region. This method is comprised essentially of the following steps:
Tiger nut cleaning and disinfection;
Grinding of the tiger nut with a hammer mill;
Water addition;
Pressing of the tiger nut, separating the pulp from the fluid;
Obtaining the natural tiger nut milk base which will be employed in the method of the invention.

### DETAILED DESCRIPTION. OF THE INVENTION

The present invention relates to a method for obtaining a cosmetic product from a tiger nut milk concentrate, characterized in that it comprises:
a) mixing in a tank sugar free-tiger nut milk with cosmetic ingredients and thermal deionized distilled water;
b) mixing in a second tank surfactants with molasses alcohol;
c) mixing the resulting products from the previous steps to produce the base concentrate;
d) applying ultrasounds to said base concentrate; and
e) adding pharmaceutically acceptable excipients.

The step a) is carried out in a tank which can be made of various materials, but preferably is made of stainless steel.

In a preferred embodiment said method is characterized in that said stainless steel tank employed in step a) comprises a propeller mixer with capacity of between 200 and 500 rpm.

In a specific embodiment, said mixture from step a) is carried out at a temperature of between 20 and 70° C. Additionally, preferably, said cosmetic ingredients employed in step a) are selected from: natural cosmetic ingredients, synthetic cosmetic ingredients, and mixtures thereof. These cosmetic ingredients or cosmetic feedstock may be any of those listed in the attachment of the Decision 96/335-CE.

In a specific embodiment, said cosmetic ingredients employed in step a) are selected from combinations of two or more of the following:
Anciano and Hamamelis distilled water;
Sclerotium gum;
water and Cyathea medullaris extract;
betaine, water, glycerin, hydrogenated Lecithum, honey and pectin;
Ivy and myrrh extracts, leucine, valine, arginine and lysine;
glycerin, water, lysolecithin, and seed oil from Perilla frutescens;
corn and soy unsaponifiable
propolis in hydroalcoholic solution,
raspberry seeds virgin vegetable oil;
rosehip virgin vegetable oil;
Argan virgin vegetable oil;
apricot nut virgin vegetable oil;
tea tree oil.

On the other hand, in a specific embodiment of the method for obtaining a cosmetic product from a tiger nut milk concentrate the tank employed in step b) is selected from a stainless steel tank and a polyester tank. Additionally, in a more preferred embodiment, the surfactants employed in step b) are selected from natural surfactants, synthetic surfactants, and mixtures thereof. In another preferred embodiment said surfactants employed in step b) are selected from anionic, cationic, non-ionic and amphoteric surfactants and mixtures thereof.

In a specific embodiment of the invention, said surfactants employed in step b) are selected from: anionic surfactants such as acyl amino acids (and salts), acyl glutamates, acyl peptides, sarcosinates, taurates, carboxylic acids and their salts, saturated chain acids, carboxylic acids esters, carboxylic acids ethers, phosphoric acid esters (and salts), sulfonic acids (and salts), acyl isotianates, alkyl aryl sulfonates, alkyl sulfonates, sulfosuccinate, sulfuric acid esters; cationic surfactants such as alkyl amines, alkyl imidazolines, ethoxylated amines, alkyl benzyl dimethyl ammonium salts, heterocyclic ammonium salts, tetraalkyl ammonium salts; amphoterics such as acyl amino acids (and derivatives), N-alkyl-amino acids; non-ionic surfactants such as alcohols and alkanolamides, amine oxides, esters.

In a particular embodiment of the method of the invention, the mixture of step c) is carried out during a time comprised between 15 and 25 minutes depending on the product which is intended to obtain, at a pH comprised between 4 and 9.

The mixture of the products obtained in steps a) and b) is carried out in a tank which may be manufactured from various materials and, which is preferably a stainless steel tank

On the other hand, in a particular embodiment of the method of the invention, the mixture of step c) is carried out during 20 minutes at a pH comprised between 4 and 9.

In step d) for ultrasounds application, the ultrasounds application time span will depend on the type of final product to obtain.

Additionally, in a specific embodiment, said step d) for ultrasounds application lasts between 20 and 60 minutes.

The present invention also relates to a tiger nut milk base concentrate for preparing a cosmetic product, characterized in that said base concentrate comprises:
tiger nut milk 100%
natural or synthetic cosmetic ingredients or mixtures,
distilled/deionized/thermal water
natural or synthetic surfactants or mixtures and
molasses alcohol.

Specific embodiments relate to a tiger nut milk base concentrate for preparing a cosmetic product, wherein said base concentrate comprises:
tiger nut milk 100%
natural or synthetic cosmetic ingredients or mixtures,
distilled/deionized/thermal water
natural or synthetic surfactants or mixtures and
molasses alcohol, and
   wherein said cosmetic ingredients are selected from combinations of two or more of the following:
Anciano and Hamamelis distilled water;
Sclerotium gum;
water and Cyathea medullaris extract;
betaine, water, glycerin, hydrogenated Lecithum, honey and pectin;
ivy and myrrh extracts, leucine, valine, arginine and lysine;
glycerin, water, lysolecithin, and seed oil from Perilla frutescens;
corn and soy unsaponifiable
propolis in hydroalcoholic solution,
raspberry seeds virgin vegetable oil;
rosehip virgin vegetable oil;
Argan virgin vegetable oil;
apricot nut virgin vegetable oil;
tea tree oil.

Generally, in the present invention, the base concentrate is the following:
concentrate/traditional liquid tiger nut milk (100%);
thermal/deionized water;
natural and synthetic surfactants,
Anciano and Hamamelis distilled water;
Sclerotium gum;
water and Cyathea medullaris extract;
betaine, water, glycerin, hydrogenated Lecithum, honey and pectin;
ivy and myrrh extracts, leucine, valine, arginine and Lysine;
glycerin, water, lysolecithin, and seed oil from Perilla frutescens;
corn and soy unsaponifiable
propolis in hydroalcoholic solution,
raspberry seeds virgin vegetable oil;
rosehip virgin vegetable oil;
Argan virgin vegetable oil;
apricot nut virgin vegetable oil;
tea tree oil.

According to preferred embodiments, said base concentrate has the following composition:
concentrate/traditional liquid tiger nut milk (100%); 30-50%
thermal/deionized water; 5-20%
natural and synthetic surfactants, 1-15%
Anciano and Hamamelis distilled water; 1-10%
Sclerotium gum; (Thickener) 1-4%
water and Cyathea medullaris extract; 1-3%
betaine, water, glycerin, hydrogenated Lecithum, honey and pectin; (Aqueous Solution of said products) 3-5%
ivy and myrrh extracts, leucine, valine, arginine and lysine; (essential amino acids) 2-6%
glycerin, water, lysolecithin, and seed oil from Perilla frutescens; (Aqueous Solution) 1-4%
corn and soy unsaponifiable 2-5%
propolis in hydroalcoholic solution, 1-8%
raspberry seeds virgin vegetable oil;
rosehip virgin vegetable oil;
Argan virgin vegetable oil;
apricot nut virgin vegetable oil;
Tea tree oil.

According to particular preferred embodiments, the base concentrate contains the mentioned oils:
raspberry seeds virgin vegetable oil;
rosehip virgin vegetable oil;
Argan virgin vegetable oil;
apricot nut virgin vegetable oil;
tea tree oil
   in combinations at concentrations providing 1-10% in the base concentrate.

According to the invention, the elemental composition of the base concentrate product is the starting point for obtaining the different finished products, which finally compose the cosmetic products and personal hygiene lines.

The present invention also relates to a cosmetic product containing the base concentrate previously defined.

According to particular embodiments, the type formulation of a finished cosmetic product contains:
natural base concentrate,
emulsifier,
active ingredients,
essential oils,
antioxidants,
preservatives,
natural dyes,
perfumes, and
sunscreens and sun protectors.

The products that are obtained from the defined base concentrate and by means of the object method of the present invention are:
Body cosmetic products: creams, skin lotions,
Facial cosmetic products
Color cosmetic products: lipstick, make
Personal hygiene products: soaps, bath gels. Deodorants, . . .
Capillary cosmetic products
Toilet soaps
Fragrances, perfumes and colognes
Line of products for sun protection

Finally the desired cosmetic product is obtained with the natural tiger nut special properties and qualities, by checking that the final product follows the next rules:
Interaction among the ingredients and components themselves (right synergism).
Interaction of the base concentrate with the remaining ingredients of the final cosmetic products.
Control of temperature, pH, redox potential and environmental factors (light, air, etc.).
Interaction of the final product with the vehicle (package).

The present invention further relates to a cosmetic product characterized in that it is obtained by the method described in the previous paragraphs and in that it comprises tiger nut milk concentrate.

The tiger nut most important properties and, consequently, those of its tiger nut milk, are due to its oleic acid, starches, essential amino acids, vitamin E, vitamin C, biotin or vitamin H, rutin or vitamin P, minerals such as phosphorous, calcium, magnesium, iron and potassium content.

### MODE OF CARRYING OUT THE INVENTION

Next, an example of specific embodiment of the invention is detailed with the object of illustrating the present invention and in no way limitative thereof.

Generally, the flow chart showed in the following illustrates an embodiment of the method of the invention:
a) mixing in a tank:
   TIGER NUT MILK 100%
   NATURAL/SYNTHETIC COSMETIC INGREDIENTS
   DISTILLED/DEIONIZED/THERMAL WATER
   Stirring at 200-500 r.p.m. between 20-70° C.
b) mixing in a second tank
   NATURAL SURFACTANTS
   SYNTHETIC SURFACTANTS
   MOLASSES ALCOHOL
   Stirring 16 hours at pH 4-9, at 200-500 r.p.m.
c) mixing the products resulting from the previous steps in a stainless steel tank in order to produce a base concentrate and applying ultrasounds.

Starting from the base concentrate previously defined and adding to it different products, a bath gel with the following composition is obtained:
Base concentrate: 10-15%
Surfactants: 30-40%
   Amphoterics: Cocamidopropyl Betaine
   Non-ionic: Glicereth-17 Cocoate
Preservative: 0.1% Usually: Propolis with pine oil
Natural dye 0.2% of a 1% solution, depending on the color that is intended to obtain, different products,
Perfume 0.5%. With a fresh note, with spring flowers, combined with wild citric notes and green tea,
Thickener: Normally is utilized salt (NaCl) 1%,
Thermal water: selected, diluted with distilled water in order to reduce the concentration of salts: Remainder up to 100%.

1-20. (canceled)

21. A tiger nut milk base concentrate for preparing a cosmetic product, characterized in that said base concentrate comprises:
tiger nut milk 100%
natural or synthetic cosmetic ingredients or mixtures thereof,
distilled/deionized/thermal water
natural or synthetic surfactants or mixtures thereof and
molasses alcohol.

22. The tiger nut milk base concentrate according to claim 21, characterized in that it comprises:
tiger nut milk 100%
natural or synthetic cosmetic ingredients or mixtures thereof,
distilled/deionized/thermal water
natural or synthetic surfactants or mixtures thereof and
molasses alcohol, and
wherein said cosmetic ingredients are selected from combinations of two or more of the following:
Anciano and Hamamelis distilled water;
Sclerotium gum;
water and Cyathea medullaris extract;
betaine, water, glycerin, hydrogenated Lecithum, honey and pectin;
ivy and myrrh extracts, leucine, valine, arginine and lysine;
glycerin, water, lysolecithin, and seed oil from Perilla frutescens;
corn and soy unsaponifiable
propolis in hydroalcoholic solution,
raspberry seeds virgin vegetable oil;
rosehip virgin vegetable oil;
Argan virgin vegetable oil;
apricot nut virgin vegetable oil;
tea tree oil.

23. The tiger nut milk base concentrate according to claim 21, characterized in that it comprises
concentrate/traditional liquid tiger nut milk 100%: 30-50%
thermal/deionized water: 5-20%
natural and synthetic surfactants: 1-15%
Anciano and Hamamelis distilled water: 1-10%
Sclerotium gum: 1-4%
water and Cyathea medullaris extract: 1-3%
betaine, water, glycerin, hydrogenated Lecithum, honey and pectin: 3-5%
ivy and myrrh extracts, Leucine, Valine, arginine and Lysine: 2-6%
glycerin, water, lysolecithin, and seed oil from Perilla frutescens: 1-4%
Corn and soy unsaponifiable: 2-5%
propolis in hydroalcoholic solution: 1-8%
raspberry seeds virgin vegetable oil;
rosehip virgin vegetable oil;
Argan virgin vegetable oil;
apricot nut virgin vegetable oil and
tea tree oil.

24. A method for obtaining a cosmetic product from the tiger nut milk concentrate defined in claim 21, characterized in that it comprises:
a) mixing in a tank sugar-free tiger nut milk with cosmetic ingredients and thermal deionized distilled water;
b) mixing in a second tank surfactants with molasses alcohol;
c) mixing the products resulting from the previous steps in a stainless steel tank in order to produce the base concentrate defined in claim 21;
d) applying ultrasounds to said base concentrate; and
e) adding pharmaceutically acceptable excipients.

25. The method for obtaining a cosmetic product from a tiger nut milk concentrate according to claim 24, characterized in that said stainless steel tank employed in step a) comprises a propeller mixer with capacity of between 200 and 500 rpm.

26. The method for obtaining a cosmetic product from a tiger nut milk concentrate according to claim 24, characterized in that said mixture from step a) is carried out at a temperature between 20 and 70° C.

27. The method for obtaining a cosmetic product from a tiger nut milk concentrate according to claim 24, characterized in that said cosmetic ingredients employed in step a) are selected from: natural cosmetic ingredients, synthetic cosmetic ingredients, and mixtures thereof.

28. The method for obtaining a cosmetic product from a tiger nut milk concentrate according to claim 24, characterized in that said cosmetic ingredients employed in step a) are selected from two or more of the following:
Anciano and Hamamelis distilled water;
Sclerotium gum;
water and Cyathea medullaris extract;
betaine, water, glycerin, hydrogenated Lecithum, honey and pectin;
ivy and myrrh extracts, leucine, valine, arginine and lysine;
glycerin, water, lysolecithin, and seed oil from Perilla frutescens;
corn and soy unsaponifiable
propolis in hydroalcoholic solution,
raspberry seeds virgin vegetable oil;
rosehip virgin vegetable oil;
Argan virgin vegetable oil;
apricot nut virgin vegetable oil and
tea tree oil.

29. The method for obtaining a cosmetic product from a tiger nut milk concentrate according to claim 24, characterized in that the tank employed in step b) is selected from a stainless steel tank and a polyester tank.

30. The method for obtaining a cosmetic product from a tiger nut milk concentrate according to claim 24, characterized in that the surfactants employed in step b) are selected from natural surfactants, synthetic surfactants, and mixtures thereof.

31. The method for obtaining a cosmetic product from a tiger nut milk concentrate according to claim 24, characterized in that the surfactants employed in step b) are selected from anionic, cationic, non-ionic and amphoteric surfactants and mixtures thereof.

32. The method for obtaining a cosmetic product from a tiger nut milk concentrate according to claim 24, characterized in that the surfactants employed in step b) are selected from acyl amino acids (and salts), acyl glutamates, acyl peptides, sarcosinates, taurates, carboxylic acids and their salts, saturated chain acids, carboxylic acids esters, carboxylic acids ethers, phosphoric acid esters (and salts), sulfonic acids (and salts), acyl isotianates, alkyl aryl sulfonates, alkyl sulfonates, sulfosuccinate, sulfuric acid esters; alkyl amines, alkyl imidazolines, ethoxylated amines, alkyl benzyl dimethyl ammonium salts, heterocyclic ammonium salts, tetraalkyl ammonium salts; acyl amino acids (and derivatives), N-alkyl-amino acids, alcohols, alcanolamides, amine oxides, esters and combinations thereof.

33. The method for obtaining a cosmetic product from a tiger nut milk concentrate according to claim 24, characterized in that the mixture from step c) is carried out during a time comprised between 15 and 25 minutes at a pH comprised between 4 and 9.

34. The method for obtaining a cosmetic product from a tiger nut milk concentrate according to claim 33, characterized in that the time of step c) is 20 minutes.

35. The method for obtaining a cosmetic product from a tiger nut milk concentrate according to claim 24, characterized in that said step d) for ultrasounds application lasts between 20 and 60 minutes.

36. A cosmetic product comprising the tiger nut milk concentrate described in claim 21.

37. The cosmetic product according to claim 36, characterized in that it comprises:
tiger nut milk base concentrate,
emulsifier,
active ingredients,
essential oils,
antioxidants,
preservatives,
natural dyes,
perfumes, and
sunscreens and sun protectors.

38. The cosmetic product according to claim 36, characterized in that it comprises a base concentrate having the following composition:
concentrate/traditional liquid tiger nut milk (100%);
thermal/deionized water;
natural and synthetic surfactants,
Anciano and Hamamelis distilled water;
Sclerotium gum;
water and Cyathea medullaris extract;
betaine, water, glycerin, hydrogenated Lecithum, honey and pectin;
ivy and myrrh extracts, leucine, valine, arginine and lysine;
glycerin, water, lysolecithin, and seed oil from Perilla frutescens;
Corn and soy unsaponifiable
propolis in hydroalcoholic solution,
raspberry seeds virgin vegetable oil;
rosehip virgin vegetable oil;
Argan virgin vegetable oil;
apricot nut virgin vegetable oil;
tea tree oil.

39. The cosmetic product according to claim 36, characterized in that it comprises a base concentrate having the following composition:
concentrate/traditional liquid tiger nut milk (100%); 30-50%
thermal/deionized water; 5-20%
natural and synthetic surfactants, 1-15%
Anciano and Hamamelis distilled water; 1-10%
Sclerotium gum 1-4%
water and Cyathea medullaris extract; 1-3%
betaine, water, glycerin, hydrogenated Lecithum, honey and pectin 3-5%
ivy and myrrh extracts, Leucine, Valine, arginine and Lysine: 2-6%
glycerin, water, lysolecithin, and seed oil from Perilla frutescens: 1-4%
Corn and soy unsaponifiable: 2-5%
propolis in hydroalcoholic solution: 1-8%
raspberry seeds virgin vegetable oil;
rosehip virgin vegetable oil;
Argan virgin vegetable oil;
apricot nut virgin vegetable oil and
tea tree oil.

40. The cosmetic product according to claim 36, characterized in that it is selected from creams, skin lotions, a facial cosmetic product, a color cosmetic product, a soap, bath gel, deodorants, a capillary cosmetic product, a toilet soap, fragrances, perfumes, colognes and products for sun protection.
